SEQUENCE LISTING (87 sequences)

SEQ ID NO: 1 - protein, 125 aa, Artificial Sequence
  Amino acid sequence
MAEVQLVESGGGLVQPGGSLRLSCAASGSRLDYYAVAWFRQAPGKEREEVSCITYSGGSTNYADSVKGRFTISRDNAKNMVYLQMNKLKPEDTARYYCTTEPYGSSCPLSGYDTWGQGTQVTVSS

SEQ ID NO: 2 - protein, 10 aa, Artificial Sequence
  CDR1
GSRLDYYAVA

SEQ ID NO: 3 - protein, 17 aa, Artificial Sequence
  CDR2
CITYSGGSTNYADSVKG

SEQ ID NO: 4 - protein, 14 aa, Artificial Sequence
  CDR3
EPYGSSCPLSGYDT

SEQ ID NO: 5 - protein, 129 aa, Artificial Sequence
  Amino acid sequence
MAQVQLVESGGGLVQPGGSLRLSCVASGETFLSYEIGWFRQAPGKEREAVSCIGGSGGSTNYADSVKGRFTISRDNAKNTVYLQMNSLKPEDTAAYYCAAGTAWPHCFGYQYPDEYDYWGQGTQVTVSS

SEQ ID NO: 6 - protein, 10 aa, Artificial Sequence
  CDR1
GETFLSYEIG

SEQ ID NO: 7 - protein, 17 aa, Artificial Sequence
  CDR2
CIGGSGGSTNYADSVKG

SEQ ID NO: 8 - protein, 18 aa, Artificial Sequence
  CDR3
GTAWPHCFGYQYPDEYDY

SEQ ID NO: 9 - protein, 128 aa, Artificial Sequence
  Amino acid sequence
MAAVQLVESGGGLVQPGGSLRLSCLVSGFIVSSYAMGWVRQVPGKGLEWVSGIYSDGRTYYADSVKGRFAISRDNAKNTVYLQMNSLKPEDTGVYYCATEPEHGSSWLGFRSADFGSWGQGTQVTVSS

SEQ ID NO: 10 - protein, 10 aa, Artificial Sequence
  CDR1
GFIVSSYAMG

SEQ ID NO: 11 - protein, 16 aa, Artificial Sequence
  CDR2
GIYSDGRTYYADSVKG

SEQ ID NO: 12 - protein, 18 aa, Artificial Sequence
  CDR3
EPEHGSSWLGFRSADFGS

SEQ ID NO: 13 - protein, 119 aa, Artificial Sequence
  Amino acid sequence
MAQVQLVESGGGLVQPGGSLRLSCAASGSISDFYAMGWYRQAPGNQRELVAAISSSGKPNYADSVKGRFTISRDNAKNTLWLQMNSLKPEDTAVYYCYADDYSAGFELRGQGALVTVSS

SEQ ID NO: 14 - protein, 10 aa, Artificial Sequence
  CDR1
GSISDFYAMG

SEQ ID NO: 15 - protein, 16 aa, Artificial Sequence
  CDR2
AISSSGKPNYADSVKG

SEQ ID NO: 16 - protein, 9 aa, Artificial Sequence
  CDR3
DDYSAGFEL

SEQ ID NO: 17 - protein, 126 aa, Artificial Sequence
  Amino acid sequence
MAELQLVESGGGLVQPGGSLTLSCAASGRFLEYYAIGWFREAPGKEREGVSCISMSAGSTNYADSVKGRFTISRDNAKNTVYLQMNSLKPEDTGVYHCASEPYGCDDGLLRGYDYWGQGTQVTVSS

SEQ ID NO: 18 - protein, 10 aa, Artificial Sequence
  CDR1
GRFLEYYAIG

SEQ ID NO: 19 - protein, 17 aa, Artificial Sequence
  CDR2
CISMSAGSTNYADSVKG

SEQ ID NO: 20 - protein, 15 aa, Artificial Sequence
  CDR3
EPYGCDDGLLRGYDY

SEQ ID NO: 21 - protein, 124 aa, Artificial Sequence
  Amino acid sequence
MAQVQLVESGGGLVQPGGSLRLSCAASGSIFSIYAMGWYRQVPEKQRELVAEITEGVNTPYAYADSVKGRFTISRDNAKNTVYLQMDSLKPEDTAVYYCHALLAGPGGGGYDYWGQGTQVTVSS

SEQ ID NO: 22 - protein, 10 aa, Artificial Sequence
  CDR1
GSIFSIYAMG

SEQ ID NO: 23 - protein, 18 aa, Artificial Sequence
  CDR2
EITEGVNTPYAYADSVKG

SEQ ID NO: 24 - protein, 12 aa, Artificial Sequence
  CDR3
LLAGPGGGGYDY

SEQ ID NO: 25 - protein, 119 aa, Artificial Sequence
  Amino acid sequence
MAEVDLVESGGGLVQPGGSLRLSCAASGSISGIYAMGWYRQAPGNQRELVAAISSGGKPNYADSVKGRFTISRDNAKNTLWLQMNSLKPEDTAVYYCYADDYSAGFELRGQGTLVTVSS

SEQ ID NO: 26 - protein, 10 aa, Artificial Sequence
  CDR1
GSISGIYAMG

SEQ ID NO: 27 - protein, 16 aa, Artificial Sequence
  CDR2
AISSGGKPNYADSVKG

SEQ ID NO: 28 - protein, 9 aa, Artificial Sequence
  CDR3
DDYSAGFEL

SEQ ID NO: 29 - protein, 126 aa, Artificial Sequence
  Amino acid sequence
MAQLQLVASGGGLVQPGGSLRLSCAASEFTDNSYFSTYAMGWARQVPGGRFEWVSAIYSDGRTYYEDSVKGRFTISRDNAKNTVHLQMNSLKPEDTAVYYCATGVAGTGWDGMDYWGKGTLVTVSS

SEQ ID NO: 30 - protein, 14 aa, Artificial Sequence
  CDR1
EFTDNSYFSTYAMG

SEQ ID NO: 31 - protein, 16 aa, Artificial Sequence
  CDR2
AIYSDGRTYYEDSVKG

SEQ ID NO: 32 - protein, 12 aa, Artificial Sequence
  CDR3
GVAGTGWDGMDY

SEQ ID NO: 33 - protein, 126 aa, Artificial Sequence
  Amino acid sequence
MAEVQLVESGGGLVQPGGSLTLSCAASGRYLEYYAIGWFREAPGKEREGVSCISMSAGSTNYADSVKGRFTVSRDNAKNTVYLQMNSLKPEDTGVYHCASEPYGCEDGLLRGYDYWGQGTQVTVSS

SEQ ID NO: 34 - protein, 10 aa, Artificial Sequence
  CDR1
GRYLEYYAIG

SEQ ID NO: 35 - protein, 17 aa, Artificial Sequence
  CDR2
CISMSAGSTNYADSVKG

SEQ ID NO: 36 - protein, 15 aa, Artificial Sequence
  CDR3
EPYGCEDGLLRGYDY

SEQ ID NO: 37 - protein, 126 aa, Artificial Sequence
  Amino acid sequence
MAQLRLVESGGGLVQPGGSLRLSCAASGFSLDYYSMGWLRQAPGKEREGISCISGSDGSTYNADSVEGRFTISRDNAKNTVYLEMASLQPEDTAVYYCARGTLYCFGPSLGGMDVWGKGTLVTVSS

SEQ ID NO: 38 - protein, 10 aa, Artificial Sequence
  CDR1
GFSLDYYSMG

SEQ ID NO: 39 - protein, 17 aa, Artificial Sequence
  CDR2
CISGSDGSTYNADSVEG

SEQ ID NO: 40 - protein, 15 aa, Artificial Sequence
  CDR3
GTLYCFGPSLGGMDV

SEQ ID NO: 41 - protein, 125 aa, Artificial Sequence
  Amino acid sequence
MAEVQLVESGGGLVQPGGSLRLSCAASGSRLDYYAVAWFRQAPGKEREEVSSITYSGGSTNYADSVKGRFTISRDNAKNMVYLQMNKLKPEDTARYYCTTEPYGSSSPLSGYDTWGQGTQVTVSS

SEQ ID NO: 42 - protein, 10 aa, Artificial Sequence
  CDR1
GSRLDYYAVA

SEQ ID NO: 43 - protein, 17 aa, Artificial Sequence
  CDR2
SITYSGGSTNYADSVKG

SEQ ID NO: 44 - protein, 14 aa, Artificial Sequence
  CDR3
EPYGSSSPLSGYDT

SEQ ID NO: 45 - protein, 123 aa, Artificial Sequence
  Amino acid sequence
MAQVQLVESGGGLVQPGGSLRLSCAVSGFTLDYYAIAWFRQAPGKEREGVSSISSSDASTWYADSVKGRFTISRDNAKNTVYLQMNSLKPEDTAVYYCADAGYQSTPLGYGSWGQGTQVTVSS

SEQ ID NO: 46 - protein, 10 aa, Artificial Sequence
  CDR1
GFTLDYYAIA

SEQ ID NO: 47 - protein, 17 aa, Artificial Sequence
  CDR2
SISSSDASTWYADSVKG

SEQ ID NO: 48 - protein, 12 aa, Artificial Sequence
  CDR3
AGYQSTPLGYGS

SEQ ID NO: 49 - protein, 113 aa, Artificial Sequence
  Amino acid sequence
MAEVQFVESGGGLVQPGGSLRLSCAASGNSFSIYAMAWYRQAPGKQRELVATITSGGHTYYADSVKGRFTLSRDNAKNTVYLQMNSLKPEDTAVYYCNVDVYWGQGTQVTVSS

SEQ ID NO: 50 - protein, 10 aa, Artificial Sequence
  CDR1
GNSFSIYAMA

SEQ ID NO: 51 - protein, 16 aa, Artificial Sequence
  CDR2
TITSGGHTYYADSVKG

SEQ ID NO: 52 - protein, 3 aa, Artificial Sequence
  CDR3
DVY

SEQ ID NO: 53 - protein, 126 aa, Artificial Sequence
  Amino acid sequence
MAQLQLVASGGGLVQPGGSLRLSCAASEFTDNSYFSTYAMGWARQVPGGRFEWVSAIYSDARTYYEDSVKGRFTISRDNAKNTVHLQMNSLKPEDTAVYYCATGVAGTGWDAMDYWGKGTLVTVSS

SEQ ID NO: 54 - protein, 14 aa, Artificial Sequence
  CDR1
EFTDNSYFSTYAMG

SEQ ID NO: 55 - protein, 16 aa, Artificial Sequence
  CDR2
AIYSDARTYYEDSVKG

SEQ ID NO: 56 - protein, 12 aa, Artificial Sequence
  CDR3
GVAGTGWDAMDY

SEQ ID NO: 57 - protein, 123 aa, Artificial Sequence
  Amino acid sequence
MAQVQLVESGGGLVQPGGSLRLSCAVSGFTLDYYAIAWFRQAPGKEREGVSCISSSDGSTWYADSVKGRFTISRDNAKNTVYLQMNSLKPEDTAVYYCADAGYQCTPLGYGSWGQGTQVTVSS

SEQ ID NO: 58 - protein, 10 aa, Artificial Sequence
  CDR1
GFTLDYYAIA

SEQ ID NO: 59 - protein, 17 aa, Artificial Sequence
  CDR2
CISSSDGSTWYADSVKG

SEQ ID NO: 60 - protein, 12 aa, Artificial Sequence
  CDR3
AGYQCTPLGYGS

SEQ ID NO: 61 - protein, 124 aa, Artificial Sequence
  Amino acid sequence
MAQVQLVESGGGLVQPGGSLRLSCAASGSTASIYAMGWYLQVPEKQRELVAEITEGVNTPYAYADSVKGRFAVSRDNAKNMVFLRMDSLKPEDTAVYYCHALLAGPGGGGYDYWGQGTLVTVSS

SEQ ID NO: 62 - protein, 10 aa, Artificial Sequence
  CDR1
GSTASIYAMG

SEQ ID NO: 63 - protein, 18 aa, Artificial Sequence
  CDR2
EITEGVNTPYAYADSVKG

SEQ ID NO: 64 - protein, 12 aa, Artificial Sequence
  CDR3
LLAGPGGGGYDY

SEQ ID NO: 65 - protein, 227 aa, Artificial Sequence
  Constant region
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 66 - protein, 464 aa, Artificial Sequence
  unnamed
MGWTLVFLFLLSVTAGVHSQVQLVQSGAEVKKPGASVKVSCKASGYTFTDYLMHWVRQAPGQGLEWIGWVDPEYGSTDYAEKFKKWVTVTRDTSISTAYMELSRLTSDDTAVYYCARDDGSYSPFDYWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKSSVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 67 - protein, 233 aa, Artificial Sequence
  unnamed
MVSSAQFLGLLLLCFQGTRCDIQMTQSPSSLSASVGDRVTVTCQASQNINKYIAWYQQKPGKAPKLLIHYTSTLESGVPSRFSGSGSGTDFTLTISSLQAEDVATYYCLQYVNLITFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 68 - protein, 123 aa, Artificial Sequence
  Amino acid sequence
EVQLVESGGGLVQPGGSLRLSCAASGSRLDYYAVAWFRQAPGKEREEVSCITYSGGSTNYADSVKGRFTISRDNAKNMVYLQMNKLKPEDTARYYCTTEPYGSSCPLSGYDTWGQGTQVTVSS

SEQ ID NO: 69 - protein, 10 aa, Artificial Sequence
  CDR1
GSRLDYYAVA

SEQ ID NO: 70 - protein, 17 aa, Artificial Sequence
  CDR2
CITYSGGSTNYADSVKG

SEQ ID NO: 71 - protein, 14 aa, Artificial Sequence
  CDR3
EPYGSSCPLSGYDT

SEQ ID NO: 72 - protein, 123 aa, Artificial Sequence
  Amino acid sequence
EVQLVESGGGLVQPGGSLRLSCAASGSRLDYYAVAWFRQAPGKEREEVSCITYSGGSTNYADSVKGRFTISRDNSKNTLYLQMNSLRAEDTAVYYCTTEPYGSSCPLSGYDTWGQGTLVTVSS

SEQ ID NO: 73 - protein, 10 aa, Artificial Sequence
  CDR1
GSRLDYYAVA

SEQ ID NO: 74 - protein, 17 aa, Artificial Sequence
  CDR2
CITYSGGSTNYADSVKG

SEQ ID NO: 75 - protein, 14 aa, Artificial Sequence
  CDR3
EPYGSSCPLSGYDT

SEQ ID NO: 76 - protein, 123 aa, Artificial Sequence
  Amino acid sequence
EVQLVESGGGLVQPGGSLRLSCAASGSRLDYYAVAWFRQAPGKEREEVSCITYSGGSTNYAESVKGRFTISRDNSKNTLYLQMNSLRAEDTAVYYCTTEPYGSSCPLSGYDTWGQGTLVTVSS

SEQ ID NO: 77 - protein, 10 aa, Artificial Sequence
  CDR1
GSRLDYYAVA

SEQ ID NO: 78 - protein, 17 aa, Artificial Sequence
  CDR2
CITYSGGSTNYAESVKG

SEQ ID NO: 79 - protein, 14 aa, Artificial Sequence
  CDR3
EPYGSSCPLSGYDT

SEQ ID NO: 80 - protein, 123 aa, Artificial Sequence
  Amino acid sequence
EVQLVESGGGLVQPGGSLRLSCAASGSRLDYYAVAWVRQAPGKELEEVSCITYSGGSTNYADSVKGRFTISRDNSKNTLYLQMNSLRAEDTAVYYCTTEPYGSSCPLSGYDTWGQGTLVTVSS

SEQ ID NO: 81 - protein, 10 aa, Artificial Sequence
  CDR1
GSRLDYYAVA

SEQ ID NO: 82 - protein, 17 aa, Artificial Sequence
  CDR2
CITYSGGSTNYADSVKG

SEQ ID NO: 83 - protein, 14 aa, Artificial Sequence
  CDR3
EPYGSSCPLSGYDT

SEQ ID NO: 84 - protein, 123 aa, Artificial Sequence
  Amino acid sequence
EVQLVESGGGLVQPGGSLRLSCAASGSRLDYYAVAWVRQAPGKELEEVSCITYSGGSTNYAESVKGRFTISRDNSKNTLYLQMNSLRAEDTAVYYCTTEPYGSSCPLSGYDTWGQGTLVTVSS

SEQ ID NO: 85 - protein, 10 aa, Artificial Sequence
  CDR1
GSRLDYYAVA

SEQ ID NO: 86 - protein, 17 aa, Artificial Sequence
  CDR2
CITYSGGSTNYAESVKG

SEQ ID NO: 87 - protein, 14 aa, Artificial Sequence
  CDR3
EPYGSSCPLSGYDT

(19) United States
(12) Patent Application Publication
(10) Pub. No.: US 2025/0043015 A1
(43) Pub. Date: Feb. 6, 2025

(54) ANTI-TNFR2 SINGLE DOMAIN ANTIBODY, AND PREPARATION METHOD THEREFOR AND APPLICATION THEREOF

(71) Applicant: SHENGHE (CHINA) BIOPHARMACEUTICAL CO., LTD., Nanjing, Jiangsu (CN)

(72) Inventors: Xiaoling JIANG, Nanjing (CN); Liusong YIN, Nanjing (CN); ZiYou SHEN, Nanjing (CN); Chongbing WU, Nanjing (CN); Jinhua ZHOU, Nanjing (CN)

(73) Assignee: SHENGHE (CHINA) BIOPHARMACEUTICAL CO., LTD., Nanjing, Jiangsu (CN)

(21) Appl. No.: 18/569,308

(22) PCT Filed: Apr. 11, 2022
(86) PCT No.: PCT/CN2022/086069

§ 371 Date: Dec. 12, 2023

(30) Foreign Application Priority Data
Jun. 30, 2021 (WO) PCT/CN2021/103550

(51) Int. Cl. C07K 16/28; A61K 39/00; A61K 45/06; A61P 35/00

(52) U.S. Cl. CPC C07K 16/2878; A61K 45/06; A61P 35/00; A61K 2039/505; C07K 2317/24; C07K 2317/52; C07K 2317/565; C07K 2317/569; C07K 2317/73; C07K 2317/76; C07K 2317/92

(57) ABSTRACT

An anti-human TNFR2 antibody or antigen-binding fragment thereof and uses and pharmaceutical compositions thereof. The anti-human TNFR2 antibody or antigen-binding fragment thereof is capable of specifically binding to TNFR2, inhibiting Treg cell proliferation, and/or promoting effector Teff cell proliferation and activation.

### TECHNICAL FIELD

The present invention belongs to the field of biomedicine, and specifically relates to an anti-TNFR2 single-domain antibody or antigen-binding fragment thereof and preparation method and use thereof.

### BACKGROUND

Cancer cells express class I major histocompatibility complex (MHC) proteins that distinguish these cells from foreign cells. In order to prevent cellular fratricide, regulatory T-cells (Treg cells) have evolved, which contain the activity of T-cells that exhibit reactivity against their “own” MHC antigens. Treg cells represent a heterogeneous class of T-cells, which can be differentiated based on their unique surface protein presentation. The most easily understood Treg cell populations include CD4+, CD25+, FoxP3+ Treg cells and CD17+ Treg cells.

Treg cells play an important role in the maintenance of peripheral tolerance, but the same biochemical features that form the basis of the ability of these cells to regulate the activity of self-reactive T cells are also used to disrupt both secondary immunotherapy and the natural immune response by curtailing the activity of tumor-reactive T lymphocytes.

Tumor necrosis factor receptor (TNFR) isoforms 1 and 2 have been identified on the surface of Treg cells as signaling molecules determining cell fate, i.e., TNFR1 and TNFR2. Among others, activation of TNFR1 enhances the cysteine asparaginase signaling cascade and terminates apoptosis in Treg cells.

Whereas, TNFR2 protein is a cell surface protein that is aberrantly expressed on the surface of a variety of human tumor cells. An anti-human TNFR2 antibody antagonist that inhibits Tregs has been reported to have greater inhibitory potency against cancer-associated Tregs cultures compared with normal peripheral Tregs. A low dose of TNFR2 antagonist was also found to kill the TNFR2-positive cell line OVCAR3. Anti-human TNFR2 antibody has the ability to turn off early intracellular phosphorylation downstream of TNFR2 prior to NF-kB-dependent cell proliferation and inhibit soluble TNFR2 secretion. The TNFR2 antagonist successfully inhibited the binding of TNFα to TNFR2 even in the presence of high concentrations of TNFα.

Second, activation of TNFR2 induces signaling through the mitogen-activated protein kinase (MAPK) signaling pathway, which orchestrates the transcription of genes that promote evasion of apoptosis and cell proliferation through TRAF2/3 signaling and NFκB mediation. Due to its role in directing cell survival and growth, TNFR2 represents an attractive target for immunodetection used to prevent tumor-reactive T lymphocytes. Therefore, therapies capable of preventing the survival and proliferation of Treg cells are currently needed for use in the treatment of targeted cell proliferative conditions (e.g., cancer) and a wide range of infectious diseases. TNFR2 can be expressed not only on cancer cells, the Treg that infiltrate tumors, but also on effector Teff cells. Studies have shown that enhancing the activity of effector T lymphocytes to target and treat a variety of diseases (e.g., cancer or autoimmune diseases) is therapeutically effective. Disease-specific immune responses can be enhanced by increasing the ability of effector T cells to suppress tumors.

Single-domain antibody (nanobody, Nb), i.e., heavy chain single-domain antibody VHH, there exists heavy chain antibody (HCAb) in camel which is naturally missing light chain, and the single-domain antibody obtained by cloning its variable region which consists of only one variable region of the heavy chain is the smallest unit of stable antigen-binding that can be obtained at present with complete function. Characterized by high stability, good water solubility, simple humanization, high targeting and strong penetration, single domain antibodies play a great function beyond imagination in immune experiments, diagnosis and therapy. Single domain antibodies are gradually becoming an emerging force in the new generation of antibody diagnosis and therapy.

At present, there is no satisfactory anti-human TNFR2 single-domain antibody, the development of a new anti-human TNFR2 single-domain antibody, so that it has not only the good performance of single-domain antibody, but also has a good performance of preventing the survival and proliferation of Treg cells, has become an urgent problem to be solved, and the antibody is required to have good specificity, blocking activity, better clinical efficacy, and simple production. It can reduce production costs and is suitable for industrial production.

### SUMMARY

In response to the shortcomings of the existing problem, the first purpose of the present invention is to provide an anti-human TNFR2 single-domain antibody.

The second purpose of the present invention is to provide a gene encoding the above-described anti-human TNFR2 single-domain antibody.

The third purpose of the present invention is to provide a vector comprising the gene encoding the above-described anti-human TNFR2 single-domain antibody.

The fourth purpose of the present invention is to provide a host cell comprising a gene vector encoding the above-described anti-human TNFR2 single-domain antibody.

The fifth purpose of the present invention is to provide a method for expressing the above-described anti-human TNFR2 single-domain antibody.

The sixth purpose of the present invention is to provide a pharmaceutical conjugate comprising the above-described anti-human TNFR2 single-domain antibody.

The seventh purpose of the present invention is to provide use of a combination comprising the above-described anti-human TNFR2 single-domain antibody and a chemotherapy in the manufacture of a drug for the treatment of cancer or an autoimmune disease.

The eighth purpose of the present invention is to provide an use comprising the above-described anti-human TNFR2 single-domain antibody.

The ninth purpose of the present invention is to provide a pharmaceutical composition comprising the anti-human TNFR2 single-domain antibody.

The technical solutions adopted by the present invention to solve its technical problems include:

1. An anti-TNFR2 antibody or antigen-binding fragment thereof, wherein the antibody or antigen-binding fragment thereof comprises complementary determining regions CDR1, CDR2 and CDR3, wherein,
(01) CDR1 is an amino acid sequence selected from any one of SEQ ID NOs: 2, 6, 10, 14, 18, 22, 26, 30, 34, 38, 42, 46, 50, 54, 58, 62, 69, 73, 77, 81, 85; or a sequence having at least 80%, 85%, 90%, 91%, 92%, 93%, 94%, 95%, 96%, 97%, 98%, 99% or more identity to an amino acid sequence of any one of SEQ ID NOs: 2, 6, 10, 14, 18, 22, 26, 30, 34, 38, 42, 46, 50, 54, 58, 62, 69, 73, 77, 81, 85; or an amino acid sequence having one or more (preferably 2 or 3) conserved amino acid mutations (preferably substitutions, insertions or deletions) compared with any amino acid sequence of SEQ ID NOs: 2, 6, 10, 14, 18, 22, 26, 30, 34, 38, 42, 46, 50, 54, 58, 62, 69, 73, 77, 81, 85;
(02) CDR2 is an amino acid sequence selected from any one of SEQ ID NOs: 3, 7, 11, 15, 19, 23, 27, 31, 35, 39, 43, 47, 51, 55, 59, 63, 70, 74, 78, 82, 86; or a sequence having at least 80%, 85%, 90%, 91%, 92%, 93%, 94%, 95%, 96%, 97%, 98%, 99% or more identity to an amino acid sequence of any one of SEQ ID NOs: 3, 7, 11, 15, 19, 23, 27, 31, 35, 39, 43, 47, 51, 55, 59, 63, 70, 74, 78, 82, 86; or an amino acid sequence having one or more (preferably 2 or 3) conserved amino acid mutations (preferably substitutions, insertions or deletions) compared with any amino acid sequence of SEQ ID NOs: 3, 7, 11, 15, 19, 23, 27, 31, 35, 39, 43, 47, 51, 55, 59, 63, 70, 74, 78, 82, 86; and
(03) CDR3 is an amino acid sequence selected from any one of SEQ ID NOs: 4, 8, 12, 16, 20, 24, 28, 32, 36, 40, 44, 48, 52, 56, 60, 64, 71, 75, 79, 83, 87; or a sequence having at least 80%, 85%, 90%, 91%, 92%, 93%, 94%, 95%, 96%, 97%, 98%, 99% or more identity to an amino acid sequence of any one of SEQ ID NOs: 4, 8, 12, 16, 20, 24, 28, 32, 36, 40, 44, 48, 52, 56, 60, 64, 71, 75, 79, 83, 87; or an amino acid sequence having one or more (preferably 2 or 3) conserved amino acid mutations (preferably substitutions, insertions or deletions) compared with any amino acid sequence of SEQ ID NOs: 4, 8, 12, 16, 20, 24, 28, 32, 36, 40, 44, 48, 52, 56, 60, 64, 71, 75, 79, 83, 87.

2. The antibody or antigen-binding fragment thereof of scheme 1, wherein,
(01) the complementary determining regions CDR1, CDR2 and CDR3 consist of the amino acid sequences SEQ ID NO: 2, SEQ ID NO: 3 and SEQ ID NO: 4, respectively;
(02) the complementary determining regions CDR1, CDR2 and CDR3 consist of the amino acid sequences SEQ ID NO: 6, SEQ ID NO: 7 and SEQ ID NO: 8, respectively;
(03) the complementary determining regions CDR1, CDR2 and CDR3 consist of the amino acid sequences SEQ ID NO: 10, SEQ ID NO: 11 and SEQ ID NO: 12, respectively;
(04) the complementary determining regions CDR1, CDR2 and CDR3 consist of the amino acid sequences SEQ ID NO: 14, SEQ ID NO: 15 and SEQ ID NO: 16, respectively;
(05) the complementary determining regions CDR1, CDR2 and CDR3 consist of the amino acid sequences SEQ ID NO: 18, SEQ ID NO: 19 and SEQ ID NO: 20, respectively;
(06) the complementary determining regions CDR1, CDR2 and CDR3 consist of the amino acid sequences SEQ ID NO: 22, SEQ ID NO: 23 and SEQ ID NO: 24, respectively;
(07) the complementary determining regions CDR1, CDR2 and CDR3 consist of the amino acid sequences SEQ ID NO: 26, SEQ ID NO: 27 and SEQ ID NO: 28, respectively;
(08) the complementary determining regions CDR1, CDR2 and CDR3 consist of the amino acid sequences SEQ ID NO: 30, SEQ ID NO: 31 and SEQ ID NO: 32, respectively;
(09) the complementary determining regions CDR1, CDR2 and CDR3 consist of the amino acid sequences SEQ ID NO: 34, SEQ ID NO: 35 and SEQ ID NO: 36, respectively;
(10) the complementary determining regions CDR1, CDR2 and CDR3 consist of the amino acid sequences SEQ ID NO: 38, SEQ ID NO: 39 and SEQ ID NO: 40, respectively;
(11) the complementary determining regions CDR1, CDR2 and CDR3 consist of the amino acid sequences SEQ ID NO: 42, SEQ ID NO: 43 and SEQ ID NO: 44, respectively;
(12) the complementary determining regions CDR1, CDR2 and CDR3 consist of the amino acid sequences SEQ ID NO: 46, SEQ ID NO: 47 and SEQ ID NO: 48, respectively;
(13) the complementary determining regions CDR1, CDR2 and CDR3 consist of the amino acid sequences SEQ ID NO: 50, SEQ ID NO: 51 and SEQ ID NO: 52, respectively;
(14) the complementary determining regions CDR1, CDR2 and CDR3 consist of the amino acid sequences SEQ ID NO: 54, SEQ ID NO: 55 and SEQ ID NO: 56, respectively;
(15) the complementary determining regions CDR1, CDR2 and CDR3 consist of the amino acid sequences SEQ ID NO: 58, SEQ ID NO: 59 and SEQ ID NO: 60, respectively;
(16) the complementary determining regions CDR1, CDR2 and CDR3 consist of the amino acid sequences SEQ ID NO: 62, SEQ ID NO: 63 and SEQ ID NO: 64, respectively;
(17) the complementary determining regions CDR1, CDR2 and CDR3 consist of the amino acid sequences SEQ ID NO: 69, SEQ ID NO: 70 and SEQ ID NO: 71, respectively;
(18) the complementary determining regions CDR1, CDR2 and CDR3 consist of the amino acid sequences SEQ ID NO: 73, SEQ ID NO: 74 and SEQ ID NO: 75, respectively;
(19) the complementary determining regions CDR1, CDR2 and CDR3 consist of the amino acid sequences SEQ ID NO: 77, SEQ ID NO: 78 and SEQ ID NO: 79, respectively;
(20) the complementary determining regions CDR1, CDR2 and CDR3 consist of the amino acid sequences SEQ ID NO: 81, SEQ ID NO: 82 and SEQ ID NO: 83, respectively; or
(21) the complementary determining regions CDR1, CDR2 and CDR3 consist of the amino acid sequences SEQ ID NO: 85, SEQ ID NO: 86 and SEQ ID NO: 87, respectively.

3. The antibody or antigen-binding fragment thereof of any one of schemes 1-2, wherein,
the sequence of the antibody or antigen-binding fragment thereof is selected from the amino acid sequence of any one of SEQ ID NO: 1, 5, 9, 13, 17, 21, 25, 29, 33, 37, 41, 45, 49, 53, 57, 61, 68, 72, 76, 80, 84,
or a sequence having at least 80%, 85%, 90%, 91%, 92%, 93%, 94%, 95%, 96%, 97%, 98%, 99% or more identity to the amino acid sequence given in SEQ ID NO: 1, 5, 9, 13, 17, 21, 25, 29, 33, 37, 41, 45, 49, 53, 57, 61, 68, 72, 76, 80, 84,
or an amino acid sequence having one or more (preferably 1, 2, 3, 4, 5, 6, 7, 8, 9, 10) conserved amino acid mutations (preferably substitutions, insertions, or deletions) as compared with any of the amino acid sequences of SEQ ID NO: 1, 5, 9, 13, 17, 21, 25, 29, 33, 37, 41, 45, 49, 53, 57, 61, 68, 72, 76, 80, 84.

4. The antibody or antigen-binding fragment thereof of any one of schemes 1-3, wherein the antibody or antigen-binding fragment thereof being a single-domain antibody VHH chain.

5. An antibody comprises one or more antibodies or antigen-binding fragments thereof of scheme 4.

6. The antibody of scheme 5, wherein the antibody comprises a monomer, a bivalent antibody, and/or a polyvalent antibody.

7. The antibody or antigen-binding fragment thereof of scheme 4, wherein the single-domain antibody VHH chain further comprises the immunoglobulin Fc region, and the immunoglobulin Fc region is selected from IgG1, IgG2, IgG3, IgG4.

8. The antibody or antigen-binding fragment thereof of scheme 4, wherein the single-domain antibody VHH chain further comprises the amino acid sequence of the immunoglobulin constant region SEQ ID NO: 65.

9. The antibody or antigen-binding fragment thereof of scheme 4, wherein it binds to human TNFR2 and inhibits the binding of TNF-α to TNFR2.

10. The antibody or antigen-binding fragment thereof of scheme 4, wherein it binds to TNFR2 of Treg and inhibits the proliferation of Treg.

11. The antibody or antigen-binding fragment thereof of scheme 4, wherein it binds to TNFR2 of Teff and promotes Teff cell proliferation and differentiation.

12. The antibody or antigen-binding fragment thereof of scheme 4, wherein it binds to TNFR2 of Teff and promotes the bioactivity of Teff.

13. The antibody or antigen-binding fragment thereof of scheme 4, wherein the dissociation constant KD between it and TNFR2 is less than 10 nM.

14. The antibody or antigen-binding fragment thereof of scheme 4, wherein the dissociation constant KD between it and TNFR2 is less than 1 nM.

15. A polynucleotide, wherein it encodes an antibody or antigen-binding fragment thereof of any one of schemes 1-3.

16. The polynucleotide of scheme 15, wherein the polynucleotide is selected from a polynucleotide sequence corresponding to the sequence of any one of SEQ ID NO: 1, 5, 9, 13, 17, 21, 25, 29, 33, 37, 41, 45, 49, 53, 57, 61, 68, 72, 76, 80, 84.

17. A recombinant vector, transgenic cell line, phage, recombinant bacterium or viral vector, wherein the recombinant vector, transgenic cell line, phage, recombinant bacterium or viral vector comprises the polynucleotide of any one of schemes 15-16.

18. An isolated host cell, wherein it contains a recombinant vector, a transgenic cell line, a phage, a recombinant bacterium or a viral vector of scheme 17.

19. The host cell of scheme 18, wherein the host cell is a prokaryotic cell.

20. The host cell of scheme 18, wherein the host cell is a eukaryotic cell.

21. The host cell of scheme 20, wherein the eukaryotic cell is a mammalian cell.

22. The host cell of scheme 21, wherein the mammalian cell is a CHO cell.

23. A method of antibody expression, wherein the recombinant vector, transgenic cell line, phage, recombinant bacterium or viral vector of scheme 17 is used to express an antibody protein in the host cell of any one of schemes 18-22.

24. Use of the antibody or antigen-binding fragment thereof of any one of schemes 1-3 in the preparation of a drug comprising the antibody or antigen-binding fragment thereof and a chemotherapy for the treatment of a tumor in a human patient, wherein the antibody and the chemotherapy are formulated to provide a therapeutic effect that is greater than the sum of the respective effects of the reagents.

25. The antibody or antigen-binding fragment thereof of any one of schemes 1-3, wherein the antibody or antigen-binding fragment thereof comprises camelid, chimeric, humanized or fully human.

26. A conjugate, wherein the conjugate comprises:
(a) the antibody or antigen-binding fragment thereof of any one of schemes 1-3, and
(b) a coupling portion selected from the group consisting of: a detectable marker, a drug, a gold nanoparticle/nanorod, a magnetic nanoparticle, a viral capsid protein or a viral particle, a radionuclide, a liposome, a chemotherapeutic agent, or a combination thereof.

27. The conjugate of scheme 26, wherein the detectable marker is a radionuclide.

28. The conjugate of scheme 26, wherein the drug is selected from the group consisting of: toxin, cytokine or enzyme.

29. A pharmaceutical composition, wherein it comprises an antibody or antigen-binding fragment thereof of any one of schemes 1-3 and a pharmaceutically acceptable carrier, diluent or excipient.

30. The pharmaceutical composition of scheme 29, wherein the composition further comprises an additional therapeutic agent.

31. The pharmaceutical composition of scheme 30, wherein the additional therapeutic agent is an immunotherapeutic agent.

32. Use of the pharmaceutical composition of scheme 29 in the preparation of a drug for the treatment of a TNFR2-associated disease, wherein the disease comprises a tumor or an autoimmune disease.

33. Use of the antibody or antigen-binding fragment thereof of any one of schemes 1-3 in the following (a), (b), (c), (d), (e) or (f):
(a) preparation of a drug that induces apoptosis in Treg cells;
(b) preparation of a drug that has Treg proliferation inhibitory properties;
(c) preparation of a drug that blocks the TNF-TNFR2 tumor signaling pathway;
(d) preparation of a drug that promotes the proliferation and differentiation and/or biological activity of Teff cells;
(e) preparation of a drug for the treatment of autoimmune diseases; or
(f) preparation of a drug that targets tumor cells for killing.

34. The use of scheme 33, wherein the tumor is selected from: ovarian cancer, melanoma, prostate cancer, intestinal cancer, gastric cancer, esophageal cancer, breast cancer, lung cancer, renal cancer, pancreatic cancer, uterine cancer, hepatocellular carcinoma, bladder cancer, cervical cancer, oral cancer, brain cancer, testicular cancer, skin cancer, colorectal cancer, malignant glioma, thyroid cancer, or a related tumor.

The present invention provides an antibody or antigen-binding fragment thereof that binds specifically to TNFR2 with high affinity, and the antibody can mediate inhibition of proliferation of Treg cells, and/or mediate proliferative and activating effects on CD8+ T cells.

The following definitions are provided to assist in understanding the invention set forth herein.

Provided herein are isolated antibodies, including murine and human antibodies, which specifically bind to a particular epitope on TNFR2 (e.g., human TNFR2). Also provided herein are methods of preparing antibodies, pharmaceutical conjugates comprising the antibodies of the present invention or antigen-binding fragments thereof, pharmaceutical compositions, and combination therapies with other therapeutic agents, and the preparation of drugs for the treatment of a wide range of diseases using the antibodies of the present invention or antigen-binding fragments thereof.

The term “single-domain antibody (sdAb)” as used herein refers to a fragment containing a single variable domain of an antibody, also known as a nanobody. They bind selectively to specific antigens as do complete antibody. It is much smaller than the 150-160 kDa mass of a complete antibody, which is only about 11-15 kDa. The first single-domain antibodies were artificially engineered from camel heavy-chain antibody and are called “VHH fragment”.

The BMK herein comprises BMK2, BMK4, BMK5, and BMK6. The sequences are obtained from: SEQ ID NO: 3 and SEQ ID NO: 4 in the Patent WO2017083525A1; SEQ ID NO: 66 and SEQ ID NO: 67 in the Patent WO2020041361A1; SEQ ID NO: 14 and SEQ ID NO: 42 in the Patent WO2020102739A1; SEQ ID NO: 150 and SEQ ID NO: 151 in the Patent WO2020061210A1, respectively.

The terms “Tumor Necrosis Factor Receptor 2”, “TNFR2”, “CD120b”, “p75”, “p75TNFR”, “p80TNF-α receptor”, “TBPII”, “TNFBR”, “TNFR1B”, “TNF-R75” and “TNFR80” are used interchangeably herein, and include all family members, mutants, alleles, fragments and species. TNFR2 mediates TNFα activity in conjunction with TNFR1. TNFR1 is a 55 kD membrane-bound protein, whereas TNFR2 is a 75 kD membrane-bound protein. TNFR2 regulates TNFα binding to TNFR1, and therefore regulates the level of TNFα necessary to stimulate NF-kB action. TNFR2 can also be cleaved (or selectively spliced) by metalloproteinases to generate soluble receptors that maintain affinity for TNFα.

The present invention also provides a composition. It contains an antibody of the invention or an active fragment thereof, and a pharmaceutically acceptable carrier. Typically, these substances may be formulated in a non-toxic, inert and pharmaceutically acceptable aqueous carrier medium, the pH of which may vary with the nature of the substance being formulated and the condition to be treated. The formulated pharmaceutical compositions can be administered by conventional routes, which include, but are not limited to intratumoral, intraperitoneal, intravenous, or topical administration.

The pharmaceutical compositions of the present invention can be used to bind to TNFR2 protein molecules directly and thus can be used to treat tumors. In addition, other therapeutic agents may be used at the same time.

The pharmaceutical compositions of the present invention contain a safe and effective amount (e.g., 0.001-99.999 wt %, preferably 0.01-90 wt %, more preferably 0.1-80 wt %) of the above described antibodies of the present invention or binding fragments thereof (or conjugates thereof) as well as a pharmaceutically acceptable carrier or excipient. Such carriers include, but are not limited to saline, buffer, glucose, water, glycerol, ethanol and combinations thereof. The pharmaceutical formulation should be compatible with the mode of administration. The pharmaceutical compositions of the present invention may be made in the form of an injection, e.g., prepared by conventional methods using saline or an aqueous solution containing dextrose and other excipients. The pharmaceutical compositions such as injections and solutions are preferably manufactured under sterile conditions. The active ingredient is administered in a therapeutically effective amount. In addition, the peptides of the present invention may be used with other therapeutic agents.

The antibody of the present invention may be used alone or in combination or conjugation with a detectable marker (for diagnostic purposes), a therapeutic agent, a PK (protein kinase) modifying portion, or any combination of these. Detectable markers for diagnostic purposes include, but are not limited to fluorescent or luminescent markers, radiolabeled markers, MRI (Magnetic Resonance Imaging) or CT (Computerized Tomography) contrast agents, or enzymes that are capable of producing a detectable product. Therapeutic agents that can be bound or coupled to antibodies of the present invention include, but are not limited to 1. Radionuclides; 2. Biotoxins; 3. Cytokines such as IL-2, etc.; 4. Gold nanoparticles/nanorods; 5. Viral particles; 6. Liposomes; 7. Magnetic nanoparticles; 8. Pre-drug-activating enzymes (e.g., DT-cardiac flavonoids (DTD) or biphenyl hydrolysate-like proteins (BPHL)); 9. Chemotherapeutic agents (e.g., cisplatin) or any form of nanoparticles; 10. Detectable markers etc.

As used herein, “isotype” refers to the class of antibody encoded by the heavy chain constant region gene (e.g., IgG1, IgG2, IgG3, IgG4, IgM, IgA1, IgA2, IgD and IgE antibody).

The term “identity” is used interchangeably with “homology” and “homologous” refers to the degree of similarity between sequences as determined by sequence comparison software such as BLAST. Methods and software for sequence comparison are well known to those skilled in the art. Modified nucleotide sequences can be obtained by making one or several amino acid or base substitutions, deletions and/or additions to a known sequence.

The term “antibody” or “immunoglobulin” used interchangeably herein includes an entire antibody and any antigen-binding fragment thereof (antigen-binding fragment) or single chain thereof. Specifically, it includes, but is not limited to, VHH fragments, nanobodies, fusion proteins, chimeric antibodies, humanized antibodies, and fully human antibodies.

The term “chimeric immunoglobulin” or “chimeric antibody” refers to an immunoglobulin or antibody whose variable region is derived from a first species and whose constant region is derived from a second species. The chimeric immunoglobulin or antibody may be constructed, for example by genetic engineering, from immunoglobulin gene segments belonging to different species.

The term “humanized antibody” means an antibody comprising at least one humanized antibody chain. The term “humanized antibody chain” refers to an antibody chain having a variable region comprising a substantially variable framework region and complementarity determination of the human antibody. Regions (CDRs) substantially derived from a non-human antibody (e.g., at least one CDR, two CDRs, or three CDRs). In some embodiments, the humanized antibody chain further comprises constant regions.

As used herein, the term “multispecific” is an artificial hybrid antibody having multiple different binding sites. Bispecific antibodies can be generated by a variety of methods, including fusion hybridomas or ligation of Fab′ fragments.

As used herein, the term “isolated” refers to antibody that is substantially free of other antibodies having different antigenic specificities. In addition, isolated antibody is typically substantially free of other cellular and/or chemical substances.

As used herein, the term “Fc region”, “Fc structural domain” or “Fc” refers to the C-terminal region of the heavy chain of an antibody. Thus, the Fc region comprises the constant region of the antibody.

As used herein, the term “antigen” is an antibody-bound entity (e.g., a protein entity or peptide), such as TNFR2.

As used herein, the terms “specific binding” and “selective binding” mean that the antibody exhibits appreciable affinity for a particular antigen or epitope and does not normally exhibit appreciable cross-reactivity with other antigens or epitopes. “Appreciable” or preferred binding includes binding at a KD of 10⁻⁷, 10⁻⁸, 10⁻⁹ or 10⁻¹⁰ M or more preferably. The KD (affinity constant) for antibody-antigen interactions represents the concentration of antibody at which 50% of the antibody and antigen molecules are bound together. Thus, at a suitable fixed antigen concentration, 50% of a higher affinity (i.e. stronger) antibody binds to the antigen molecule at a lower antibody concentration compared with the antibody concentration required to achieve the same percentage of binding with a lower affinity antibody. Thus, a lower KD value indicates a higher (stronger) affinity. As used herein, a “better” affinity is stronger than its affinity and has a lower value of KD of 10⁻⁷ M, and thus a better affinity compared with a KD of 10⁻⁶ M. It is generally preferred to have a KD value of less than 10⁻⁷ M, and therefore preferably greater than 10⁻⁸ M. Intermediate values as described herein may also be considered, and the preferred binding affinity may be expressed as a range of affinities, e.g., from 10⁻⁷ to 10⁻¹² M for the anti-TNFR2 antibodies disclosed herein, and more preferably from 10⁻⁸ to 10⁻¹² M. Antibodies that “do not exhibit significant cross-reactivity” or “do not bind with physiologically relevant affinity” are antibodies that do not significantly bind to the antibody. Off-target antigens (e.g., non-TNFR2 proteins) or epitopes. Specific or selective binding can be determined according to any art of the art. Recognized methods for determining such binding include, for example, according to Scatchard analysis, biomolecule interaction analysis, biofilm layer interferometry and/or competitive (competitive) binding assays.

As used herein, the term “epitope” means an antigenic determinant cluster in an antigen and refers to an antigenic site bound by a structural domain of an antigen-binding molecule comprising an antibody variable region as disclosed in this specification. Thus, an epitope can be defined, for example, on the basis of its structure. Alternatively, the epitope may be defined based on the antigen-binding activity in the antigen-binding molecule that recognize the epitope. When the antigen is a peptide or polypeptide, the epitope may be designated by the amino acid residues forming the epitope. Alternatively, when the epitope is a sugar chain, the epitope may be defined by its specific sugar chain structure.

A linear epitope is an epitope containing a primary amino acid sequence for which is recognized. Such linear epitopes typically contain at least three, most commonly at least five, such as about 8 to 10 or 6 to 20 amino acids in their particular sequence.

In contrast to linear epitopes, “conformational epitopes” are epitopes in which the primary amino acid sequence containing the epitope is not the sole determinant of the recognized epitope (e.g. the primary amino acid sequence of the conformational epitope is not necessarily recognized by the epitope-qualifying antibody). A conformational epitope may contain a greater number of amino acids than a linear epitope. Conformational epitopes recognize the three-dimensional structure of the peptide or protein recognized by the antibody. For example, when a protein molecule folds and forms a three-dimensional structure, the amino acids and/or peptide backbone forming the conformational epitope become aligned and the epitope can be recognized by the antibody. Methods for determining epitope conformation include, for example, but are not limited to, X-ray crystallography, two-dimensional nuclear magnetic resonance, site-specific spin labelling and electron paramagnetic resonance.

As used herein, the term “vector” refers to a nucleic acid molecule capable of transporting another nucleic acid to which it has been attached. One type of vector is a “plasmid”, which refers to a circular double-stranded DNA loop. Other DNA fragments can be attached to it. Another type of vector is the viral vector, in which additional DNA fragments can be attached to the viral genome. Some vectors are capable of replicating autonomously in the host cell into which they are introduced (e.g., bacterial vectors with replicating bacterial origins and free-living mammalian vectors). Other vectors (e.g., non-exogenous mammalian vectors) can be integrated into the genome of the host cell and then introduced into the host cell, thereby replicating with the host genome. In addition, some vectors are capable of directing the expression of the genes they express. These vectors are referred to herein as “recombinant expression vectors” (or simply “expression vectors”). Typically, expression vectors useful in recombinant DNA technology are usually in the form of plasmids. The terms “plasmid” and “vector” are used interchangeably. However, other forms of expression vectors with equivalent functions are also contemplated, such as viral vectors (e.g., replication-defective retroviruses, adenoviruses and adeno-associated viruses).

As used herein, the term “inhibition” refers to any statistically significant reduction in biological activity, including partial and complete blockage of that activity. For example, “inhibition” may denote a statistically significant decrease of about 10%, 20%, 30%, 40%, 50%, 60%, 70%, 80%, 90%, 95%, 96%, 97%, 98%, 99% or 100% of the biological activity.

As used herein, the term “activation” refers to any statistically significant activation of the biological activity of a cell, e.g., “activation” may denote a statistically significant increase of about 1%, 10%, 20%, 30%, 40%, 50%, 60%, 70%, 80%, 90%, 100%, 200%, 500%, 1,000%, 10,000%, and more of biological activity.

As used herein, the phrase “inhibits binding of TNFR2 ligand to TNFR2” means that the antibody statistically significantly reduces the ability of the TNFR2 ligand (e.g., TNFα) to bind to TNFR2 relative to the absence of the TNFR2 antibody. In other words, in the presence of an antibody, the amount of TNFR2 ligand that binds to TNFR2 is statistically significantly reduced relative to the control (no antibody). In the presence of the anti-TNFR2 antibody as disclosed herein, the amount of TNFR2 ligand binding to TNFR2 can be reduced by at least about 10%, or at least about 20%, or at least about 30%, or at least about 40%, or at least about 50%, or at least about 60%, or at least about 70%, or at least about 80%, or at least about 90%, or about 100%. The reduction in TNFR2 ligand binding can be measured using techniques accepted in the art that measure the level of binding of labeled TNFR2 ligand (e.g., radiolabeled TNFα) to cells expressing TNFR2 in the presence or absence of (control) antibody.

As used herein, the term “inhibition of tumor growth” includes any measurable reduction in tumor growth, e.g., an inhibition of tumor growth of at least about 10%, e.g., at least about 20%, at least about 30%, at least about 40%, at least about 50%, at least about 60%, at least about 70%, at least about 80%, at least about 90%, at least about 99%, or about 100%.

As used herein, the term “treatment” refers to the therapeutic or prophylactic measures described herein. Methods of “treatment” are intended for administration to a subject or subjects susceptible to tumors or cancers. An anti-TNFR2 antibody (e.g., an anti-human TNFR2 antibody) is described herein for the purpose of preventing, curing, delaying, or alleviating one or more symptoms of a disease or condition or recurrent disease or condition, or for the purpose of prolonging the survival of a subject for a prolonged period of time in the absence of such treatment.

As used herein, the term “variable fragment (Fv)” refers to the smallest unit of an antibody-derived antigen-binding structural domain, which consists of a pair of antibody light chain variable regions (VL) and antibody heavy chain variable regions (VH). In 1988, Skerra and Pluckthun found that homologous and active antibodies could be prepared from the periplasmic fraction of E. coli by inserting an antibody gene downstream of a bacterial signal sequence and inducing expression of the gene in E. coli. In Fv prepared from the periplasmic fraction, VH binds to VL in an antigen-binding manner.

Herein, the Fv preferably comprises, for example, a pair of Fvs acting as antigen-binding molecules, etc., comprising: scFv, a single-chain antibody, and sc(Fv)2.

As used herein, the terms “scFv”, “single chain antibody” and “sc(Fv)2” all refer to an antibody fragment of a single polypeptide chain containing variable regions derived from the heavy and light chains, but no constant regions. Typically, single-chain antibodies also contain a polypeptide junction between the VH and VL structural domains, which enables the formation of the desired structure thought to permit antigen binding.

As used herein, the term “CHOK1-hTNFR2” refers to a cell constructed by certain techniques. Specific construction methods include, but are not limited to the following methods. Detailed steps: (1) Gene synthesis and molecular construction: the sequence of human TNFR2 protein was codon optimized and synthesised by GENEWIZ company. The synthesised gene was further subcloned into the modified pSBbi-GB vector. (2) Transient transfection: 1.0×10⁶ CHOK1 cells with higher than 95% survivability were prepared in T25 culture flasks one day prior to transfection, and transfection was initiated at 70%-90% confluence. The target gene was mixed with the transposase plasmid pCMV(CAT)T7-SB100 in 9:1 ratio, followed by the addition of the Lipofectamine® 2000 DNA Transfection Reagent mix, and then the cell culture medium was added. The cells were kept in a 37° C. thermostat while the CO₂ concentration was maintained at 8%. After 48 hours of incubation, 2×10⁵ cells were firstly detached by trypsin and then centrifuged at 1,500 rpm, 4° C. for 4 minutes for FACS detection of TNFR2 expression levels. (3) Stable cell pool and cell line production. After transient transfection with FACS to confirm TNFR2 expression, the cell pool was further cultured in a medium containing pyrifoside for stable cell pool selection. The concentration of pyrifosin is 10 μg/mL. Change the culture medium every 2-3 days. After recovering from 2 to 3 weeks of antibiotic selection, a stable cell pool will be generated. Further generation of stable single cell lines through BD FACS Melody sorting. Firstly, count the cells and measure their vitality. After incubation with primary and secondary antibodies, individual cells were sorted into 96 well plates and cultured in an incubator until clones could be observed. After FACS detection, the highly expressed monoclonal clones were amplified and stored in the library. (4) FACS assay. Transiently transfected cells were transferred to 96 well U-bottom plates (Corning-3799) at a density of 2×10⁵ cells/well. Anti-TNFR2 antibody was diluted at 2 μg/mL in 2% FBS/1×PBS at 100 μL per well and then incubated for 1 hour at 4° C. Cells were washed twice and resuspended in 100 μL of 2% BSA/1×PBS. Secondary antibody (goat anti-human IgGFc-Alexa647) was diluted 1:500 in 2% FBS/1×PBS, 100 μL per well, followed by incubation at 4° C. for 30 minutes. Cells were washed twice and resuspended in 100 μL of 2% FBS/1×PBS. Fluorescence was measured by flow cytometry (BD Canto II) and analysed by FlowJo. (5) Stability assay. Cell libraries or cell lines passaged for more than 3 weeks after P1 are used for stability assay. FACS is used for stability confirmation.

### BRIEF DESCRIPTION OF THE DRAWINGS

FIG. 1 shows a schematic diagram of the FACS binding activity of the chimeric antibody to human TNFR2;

FIG. 2 shows a schematic diagram of the ligand blocking assay with anti-human TNFR2 antibody;

FIG. 3 shows a schematic diagram of the first set of anti-human TNFR2 antibody inhibition of Treg proliferation experiments;

FIG. 4 shows a schematic diagram of the second set of anti-human TNFR2 antibody inhibition of Treg proliferation experiments;

FIG. 5 shows a schematic diagram of in vivo anti-tumor efficacy evaluation of chimeric antibody.

### DETAILED DESCRIPTION

The following embodiments facilitate a better understanding of the invention, but do not limit the invention. The experimental methods in the following embodiments are conventional methods, if not otherwise specified. The test materials used in the following embodiments are, unless otherwise stated, purchased from a conventional biochemical reagent shop.

### Example 1 Animal Immunization

Recombinant human TNFR2-His Tag protein (Sino, Cat: 10417-H08H) was used as immunogen to immunize alpacas. Negative serum was taken 1 day in advance, and the first immunization was performed by multipoint injection of 200 g of recombinant human TNFR2-His Tag protein fully emulsified with Fuchs' complete adjuvant by neck immunization and upper hind leg immunization, and the second immunization was performed by multipoint injection of 100 μg of recombinant human TNFR2-His Tag protein fully emulsified with Fuchs' complete adjuvant by neck immunization and upper hind leg immunization on the 7th day. Third, fourth, fifth, sixth, seventh and eighth immunizations were performed by injecting 100 μg of immunogen in the same manner as the second immunization every two weeks. After 50 days, antibody serum titers were assessed by testing sera collected from venous blood collection in recombinant human TNFR2-His Tag protein-encapsulated ELISA plates at various dilutions ranging from 1:100 to 1:6,000,000. When the titer result meets the requirements and anti-human TNFR2 antibody is detected at dilutions >1:100,000, the blood could be collected for banking.

### Example 2 Phage Library Construction

100 mL of blood was collected from the jugular vein 10 days after the seventh immunization, and PBMC (peripheral blood mononuclear cells) were isolated using lymphocyte isolation solution. Total RNA from PBMC was extracted and reverse-transcribed by PrimeScript™ II 1st Strand cDNA Synthesis Kit (Takara, item No. 6210A) with a total of 5 μg RNA. The cDNA stock solution was mixed in equal proportions and diluted 5 times, and 5.0 μL was added for the first round of amplification, and the amplified product was gelled for recovery. The recovered product was used as the template for the second round of amplification, and the amplified product was gelled for recovery as the target fragment. The vector and the target fragment were enzymatically cut with SfiI, and the target fragment was recovered after overnight digestion at 50° C. The link molar ratio was Vector:VHH=1:3. A total of 10 times of electric transformation were performed. Immediately after the shock, 1 mL of 2YT medium (preheated at 37° C.) was added to the shock cup for resuscitation, the shock products were sucked out and the shock cup was washed with 2YT medium. A total of 100 mL resuscitation products were obtained, and resuscitation was performed at 37° C. and 180 rpm for 45 min. Gradient dilution of 100 μL was taken to 10⁻³ and 10⁻⁴ to determine the number of converters in the library, and coated on a 90 mm plate, and the rest was centrifuged. Then, 8 mL of 2YT was added to resuspend, and coated on eight 200 mm plates. On the second day, the number of converters in the library was measured and the capacity of the library was calculated.

The bacterial library was inserted into 2×300 mL 2YT+A+G (Amp: 100 μg/ml, Glu: 1%) medium until its initial OD600=0.1-0.2, and incubated at 37° C. and 230 rpm until OD600=0.8 or above. The auxiliary phage M13K07 was added according to the OD600 value (auxiliary phage:bacteria=20:1). After adding M13K07, it was mixed well and left to stand at 37° C. for 30 min. It was shaken slowly at 37° C. and 180 rpm for 30 min. It was centrifuged at 5000 rpm for 10 min, and then the supernatant was discarded fully. The precipitate was resuspended with an equal volume of 2YT+A+K (Amp: 100 μg/ml, Kan: 50 μg/mL) medium at 30° C. and 220 rpm overnight. Overnight cultures were centrifuged at 4° C. and 10,000 rpm for 20 min, and then the supernatant was collected, and the precipitate was discarded. The centrifuge cartridge was replaced, and then centrifuged at 4° C. and 10000 rpm for 20 min, and the supernatant was collected. PEG8000/NaCl was added by ⅕ of the supernatant volume, and then it was mixed well and precipitated for more than 2 hours in ice bath. It was centrifuged at 4° C. and 10,000 rpm for 20 min, and then the supernatant was discarded and centrifuged empty once to fully remove the supernatant. 1 mL of 1×PBS was used to suspend the precipitate, and PEG8000/NaCl was added by ⅕ of the supernatant volume to precipitate again for 1 h. It was centrifuged at 4° C. and 12000 rpm for 10 min, and then supernatant was discarded. According to the amount of precipitate, 1×PBS was added to resuspend the precipitate. 100% glycerol was added until the final concentration was 50%, mixed well and distributed into 1.5 mL EP tubes and stored at −80° C. 10 L of library phage was taken and diluted with 2YT gradient. 10 μL was taken from 10⁻⁸ and 10⁻⁹ tubes and added to 90 μL of TG1 bacteriophage solution, and mixed gently. Standing at 37° C. for 15 min, Amp resistant plates were coated separately and incubated overnight. The next day, titre of cloned metal phage library on the titre plate was calculated.

### Example 3 Phage Library Screening for Positive Antibody

The target molecule human TNFR2-His Tag protein (Sino, Cat: 10417-H08H) was diluted with carbonate buffer at pH 9.6 to a final concentration of 5 μg/mL, and added to the enzyme-labeled wells at 100 μL/well. Each target molecule was coated with 8 wells (It was coated with 4 wells in the second round of screening, and it was coated with 2 wells in each of the third and fourth rounds of screening), and the wells were coated at 4° C. overnight. The coating solution was discarded, and washed 3 times with PBS. 300 μL of 3% BSA-PBS blocking solution was added to each well, and blocked at 37° C. for 1 h. The wells were washed 3 times with PBS, and then 100 μL of phage library was added, and they were incubated at 37° C. for 1 h. The unbound phage was aspirated, and then the wells were washed with PBST for 6 times, and washed with PBS for 2 times. 100 μL of Gly-HCl eluate was added and incubated at 37° C. for 8 min to elute specifically bound phage. The eluate was transferred to a 1.5 mL sterile centrifuge tube and quickly neutralized with 10 μL of Tris-HCl neutralization buffer. 10 μL was taken for gradient dilution, and then the titer was measured, and the panning recovery was calculated. The remaining eluate was mixed, amplified and purified for the next round of affinity panning.

The panning eluate was mixed with 5 mL of E. coli TG1 culture in the pre-logarithmic growth phase, and allowed to stand for 30 min at 37° C., and incubated at 220 r/min with shaking for 30 min. It was centrifuged at 1000 g for 15 min, and then the supernatant was removed, and coated with 500 μL of 2×YT resuspension onto a 200 mm 2×YT-GA plate. The bacteria were scraped with 10 mL of 2×YT liquid medium, and 500 μL of suspension was added into 50 mL of 2×YT liquid medium, and then it was shaken at 37° C. for 30 min. M13K07 auxiliary phage was added at the ratio of cell:phage=1:20, and allowed to stand for 30 min at 37° C., and shaken at 220 r/min for 30 min. The cultures were distributed in centrifuge tubes, and centrifuged at 25° C. and 5000 r/min for 10 min. The cell precipitates were resuspended in 50 mL of 2×YT-AK liquid medium, and incubated overnight at 30° C. and 230 r/min with shaking. Overnight cultures were centrifuged at 4° C. and 10000 r/min for 20 min, and the supernatant was transferred to a new centrifuge tube. PEG/NaCl was added by ⅕ of the supernatant volume, and then it was mixed well and placed at 4° C. for more than 2 h. It was centrifuged at 4° C. and 10000 r/min for 20 min, and the supernatant was removed. The precipitate was resuspended in 1 mL PBS, and then PEG/NaCl was added by ⅕ of the supernatant volume. It was mixed well and placed at 4° C. for more than 1 h. It was centrifuged at 4° C. and 12000 r/min for 2 min. The supernatant was removed, and then the precipitate was suspended in 200 μL PBS, and the amplification product was obtained. The titer was determined for the next round of panning or analysis.

From the plate of panning eluent titre, 96 clones (NO. 1-96) were randomly selected from the plate of the second round of titre assay. 96 clones (NO. 97-192) were randomly selected from the first round of titre plate, and then inoculated in 1 mL of 2×YT-A, and incubated at 37° C. and 230 r/min with shaking for 8 hour. 200 μL of the above culture was taken, and M13K07 phage was added at the ratio of cell:phage=1:20. It was allowed to stand for 15 min at 37° C., and incubated at 220 r/min with shaking for 45 min. 800 μL of 2×YT-AK was added, and incubated at 30° C. with vigorous shaking overnight. The second day, it was centrifuged at 12,000 rpm for 2 min, and then supernatant was taken and used for monoclonal ELISA identification.

The target molecule TNFR2 antigen was diluted with carbonate buffer at pH 9.6 to a final concentration of 2 μg/mL, and added to the enzyme-labeled wells per 100 μL/well, and coated at 4° C. overnight. The coating solution was discarded and washed 3 times with PBST. 300 μL of 5% skimmed milk was added to each well, and the wells were blocked at 37° C. for 1 hour. The wells were washed 3 times with PBST, and 50 μL of phage culture supernatant and 50 μL of 5% skimmed milk were added to each well, and the wells were incubated at 37° C. for 1 h. The wells were washed 5 times with PBST, and an anti-M13 antibody labeled with horseradish peroxidase was added (diluted with PBS at 1:10,000) at 100 μL/well and treated at 37° C. for 1 h. The plate was washed 6 times with PBST. TMB color developing fluid was added at 100 μL/well for color development at 37° C. for 7 min. Termination solution was added at 50 μL/well to terminate the reaction, and the optical density was measured at 450 nm.

### Example 4 Sequencing of Antibody Gene Sequence

The sequences obtained from phage library screening were subjected to antibody gene sequencing. 16 antibodies were selected, and their amino acid/nucleotide sequences were as follows:

TABLE 1
Correspondence between number and sequence

| Antibody | Sequence |
|---|---|
| Antibody 01 | SEQ ID NO: 01 |
| Antibody 02 | SEQ ID NO: 05 |
| Antibody 03 | SEQ ID NO: 09 |
| Antibody 04 | SEQ ID NO: 13 |
| Antibody 05 | SEQ ID NO: 17 |
| Antibody 06 | SEQ ID NO: 21 |
| Antibody 07 | SEQ ID NO: 25 |
| Antibody 08 | SEQ ID NO: 29 |
| Antibody 09 | SEQ ID NO: 33 |
| Antibody 10 | SEQ ID NO: 37 |
| Antibody 11 | SEQ ID NO: 41 |
| Antibody 12 | SEQ ID NO: 45 |
| Antibody 13 | SEQ ID NO: 49 |
| Antibody 14 | SEQ ID NO: 53 |
| Antibody 15 | SEQ ID NO: 57 |
| Antibody 16 | SEQ ID NO: 61 |

### Example 5 Construction and Expression of Chimeric Antibodies

The sequences obtained from phage library screening were subjected to antibody gene sequencing, and the antibody fragments obtained from sequencing were subjected to gene synthesis, and constructed into the framework of human IgG1 (SEQ ID NO: 65). Mammalian cell expression plasmid was constructed by inserting into pcDNA3.1-G418 vector using molecular cloning technique. pcDNA3.1-G418 vector contained promoter CMV Promoter, eukaryotic screening marker G418 tag and prokaryotic screening tag Ampicillin. Nucleotide sequences of antibody expression light chain and heavy chain were obtained by gene synthesis, and the vector and target fragment were double-digested with HindIII and XhoI, recovered and then enzymatically ligated by DNA ligase and transformed into E. coli competent cell DH5α. Positive clones were picked out, plasmid extracted and enzymatically verified, and recombinant plasmids containing were obtained.

Recombinant plasmids containing each of the above target genes were transformed into E. coli competent cells DH5α according to the method described in the Guidelines for Molecular Cloning Experiments (2002, Science Press), and the transformed bacteria were coated and cultured on the LB plate containing 100 μg/mL of ampicillin, and the plasmid were selected and cloned into the liquid LB medium for culture. The bacteria was shaken at 260 rpm for 14 hours. The plasmids were extracted by the endotoxin-free plasmid macroabsorbent kit, and solubilized in sterile water, and the concentration was determined by the nucleic acid protein quantifier.

ExpiCHO was cultured to a cell density of 6×10⁶ cells/mL at 37° C., 8% CO₂, and 100 rpm. Recombinant plasmids were transfected into the above cells by liposomes at a concentration of 1 μg/mL, and the concentration of liposomes was determined according to the ExpiCHO™ Expression System kit, and cultured at 32° C., 5% CO₂, and 100 rpm for 7-10 days. Feeding was given once after 18-22 h of transfection and between day 5-8, respectively. The above culture product was centrifuged at 4000 rpm, and filtered through a 0.22 μm filter membrane and the supernatant of the medium was collected, and the antibodies were purified by using Protein A.

### Example 6 FACS Binding Activity of Chimeric Antibody to Human TNFR2

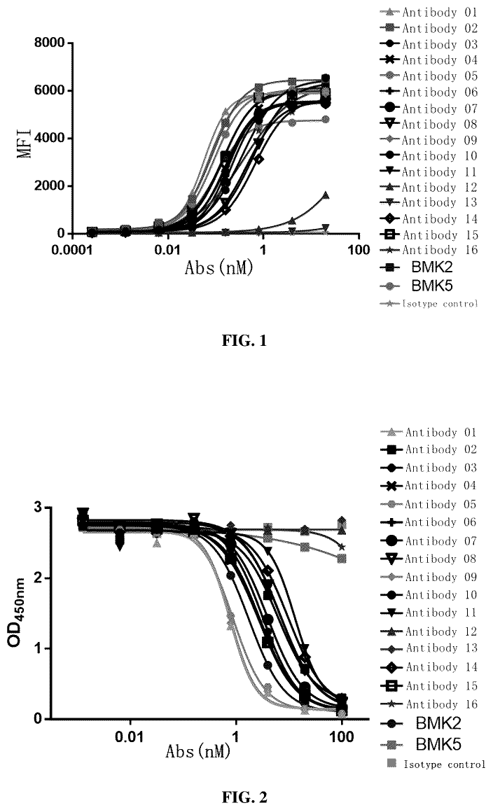

CHOK1-hTNFR2 cells were plated at 1×10⁵/well (containing 1% BSA). 100 μL of candidate antibodies were added at a concentration of 20 nM and 5-fold diluted in 8 gradients (containing 1% BSA). Cells were incubated at 4° C. for 1 hour and then washed twice with excess FACS buffer. Cells were resuspended in 100 μL of FACS buffer, and 100 μL of goat anti-human IgG Fc-AF647 (1:500) containing 1% BSA was added, incubated at 4° C. without light for 30 minutes and washed twice with excess FACS buffer. Cells were fixed in fixation buffer and subsequently analyzed by flow cytometry. The FACS method screened for candidate antibodies with high specific binding activity to human TNFR2. As shown in Table 2 and FIG. 1, and according to the experimental results, 4 antibodies have better binding activity than BMK2, and 10 antibodies have equivalent binding activity to BMK2.

TABLE 2
FACS binding activity of chimeric antibodies to human TNFR2

| | | | Parental | |
|---|---|---|---|---|
| | | Maximum | cell (20 nM Ab | |
| | | average | average | |
| | EC50 | fluorescence | fluorescence | |
| Antibody | (nM) | value | value) | NC/BL |
| Antibody 01 | 0.056 | 6003 | 48.6 | 44.7/34.8 |
| Antibody 02 | 0.084 | 6512 | 49.1 | |
| Antibody 05 | 0.091 | 6026 | 59 | |
| Antibody 09 | 0.070 | 5917 | 53.6 | |
| Antibody 10 | 0.359 | 6542 | 49.5 | |
| Antibody 11 | 0.608 | 6225 | 56.6 | |
| Antibody 12 | >20 | 1645 | 60.3 | |
| Antibody 15 | 0.139 | 5893 | 50.8 | |
| Antibody 03 | 0.171 | 5570 | 60.2 | 47.5/32.9 |
| Antibody 04 | 0.161 | 5513 | 55.7 | |
| Antibody 06 | 0.182 | 5535 | 56.8 | |
| Antibody 07 | 0.214 | 5481 | 72.3 | |
| Antibody 08 | 0.441 | 5461 | 48.3 | |
| Antibody 13 | >20 | 239 | 56.7 | |
| Antibody 14 | 0.658 | 5492 | 56.8 | |
| Antibody 16 | 0.392 | 6306 | 50.7 | |
| BMK2 | 0.139 | 6152 | 53.2 | |
| BMK5 | 0.098 | 4803 | 57.2 | |
| Isotype control | >20 | 60.6 | 51.4 | |

### Example 7 Ligand Blocking Assay

Anti-His antibody (1 μg/mL) was coated at 50 μL/well, incubated at 4° C. overnight and washed 3 times with PBST. 2% BSA was added at 200 μL/well, incubated at room temperature for 1 hour and washed 3 times with PBST. 0.25 μg/mL (containing 2% BSA) of recombinant human TNFR2-His Tag protein (Sino; Cat: 10417-H08H) was added at 50 μL/well, incubated for 1 hour at room temperature and washed 3 times with PBST. Primary antibody (isotype control or candidate antibody) was added at a concentration of 100 nM and diluted 5-fold in 8 gradients with a solution containing 1.6 nM recombinant human TNF-alpha-Biotin protein (Sino; Cat: 10602-HANE-B) and 2% BSA, incubated for 2 hours at room temperature and washed 3 times with PBST. Secondary antibody SA-HRP (1:5000) containing 2% BSA was added at 50 μL/well, incubated for 1 hour at room temperature and washed 3 times with PBST.

After washing, TMB developed the color for 10 min, and the OD450 reading was determined by the Enzyme Labeler after the display was terminated. As shown in Table 3 and FIG. 2, 3 antibodies show better ligand blocking ability than BMK2, and 10 antibodies show equivalent ligand blocking ability to BMK2.

TABLE 3
Ligand blocking assay

| | IC50 | Inhibition | PC (1.6 nM | |
|---|---|---|---|---|
| Antibody | (nM) | rate (%) | hTNFR2L1-biotin) | NC/BL |
| Antibody 01 | 0.78 | 197.3 | 2.818 | 0.062/0.063 |
| Antibody 02 | 6.11 | 89.3 | | |
| Antibody 05 | 0.89 | 97.1 | | |
| Antibody 09 | 0.79 | 97.2 | | |
| Antibody 10 | 7.00 | 91.8 | | |
| Antibody 11 | 13.1 | 90.0 | | |
| Antibody 12 | >100 | 14.7 | | |
| Antibody 15 | 2.65 | 195.5 | | |
| Antibody 03 | 3.23 | 95.3 | 2.618 | 0.067/0.063 |
| Antibody 04 | 2.64 | 94.8 | | |
| Antibody 06 | 2.79 | 95.0 | | |
| Antibody 07 | 3.93 | 93.6 | | |
| Antibody 08 | 6.77 | 90.9 | | |
| Antibody 13 | >100 | −0.3 | | |
| Antibody 14 | 9.67 | 89.0 | | |
| Antibody 16 | >100 | 6.7 | | |
| BMK2 | 1.74 | 96.3 | | |
| BMK5 | >100 | 17.1 | | |
| Isotype control | >100 | 2.5 | | |

### Example 8 Inhibition of Treg Proliferation by Anti-Human TNFR2 Antibody

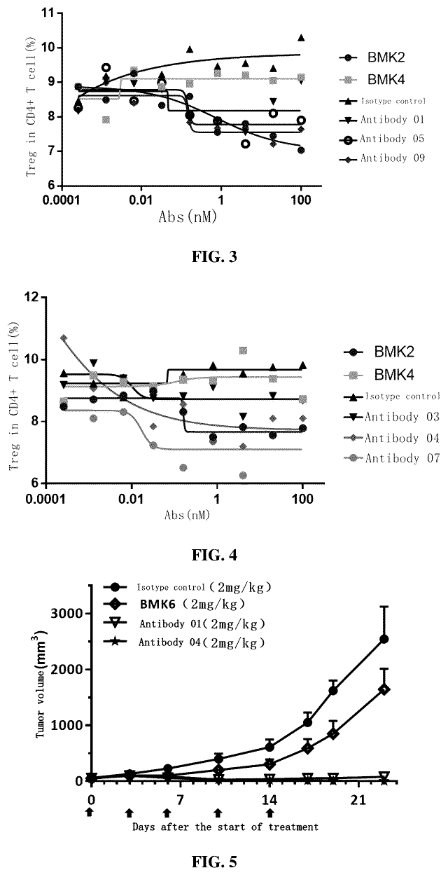

CD4+ T cells were isolated from PBMC of healthy volunteers. 200 nM of candidate antagonistic antibody was 5-fold diluted with 9 gradients, and 200 U/mL IL-2 and 20 ng/mL recombinant human TNF-alpha were added. CD4+ T cells were added at 2×10⁵/well, and incubated at 37 WC for 72 hours. Staining was performed using PE-an-CD25 (1:50) and Alexa Fluor 488-anti-Foxp3 (1:50) and analyzed by counting using a BD FACS Canto II flow cytometer. According to the experimental results, all six antibodies showed excellent proliferation inhibition ability of Treg cells. The results in Table 4 are shown in FIG. 3, and the results in Table 5 are shown in FIG. 4.

TABLE 4
Inhibition of Treg proliferation by anti-human TNFR2 antibody

| | | | | | IL-2 + |
|---|---|---|---|---|---|
| | | Minimum | Maximum | Only IL-2 Treg | hTNFR2L1 |
| | | Treg in CD4+ | Treg in CD4+ | in CD4+ T cell | (Treg in CD4+ T |
| Antibody | IC50 (nM) | T cell (%) | T cell (%) | (%)) | cell (%)) |
| BMK2 | 0.7754 | 7.03 | 9.25 | 6.96 | 9.41 |
| BMK4 | >100 | 7.92 | 9.35 | | |
| Isotype | >100 | 8.47 | 10.30 | | |
| control | | | | | |
| Antibody 01 | ~0.04552 | 7.55 | 9.05 | | |
| Antibody 05 | ~0.1453 | 7.22 | 9.43 | | |
| Antibody 09 | ~0.1457 | 7.21 | 8.98 | | |

TABLE 5
Inhibition of Treg proliferation by anti-human TNFR2 antibody

| | | | | | IL-2 + |
|---|---|---|---|---|---|
| | | Minimum | Maximum | Only IL-2 | hTNFR2L1 |
| | | Treg in CD4+ | Treg in CD4+ | ( Treg in CD4+ | (Treg in CD4+ T |
| Antibody | IC50 (nM) | T cell (%) | T cell (%) | T cell (%)) | cell (%)) |
| BMK2 | ~0.1623 | 7.50 | 8.98 | 6.27 | 8.73 |
| BMK4 | >100 | 8.65 | 10.30 | | |
| Isotype | >100 | 8.79 | 9.83 | | |
| control | | | | | |
| Antibody 03 | 0.01022 | 8.16 | 9.88 | | |
| Antibody 04 | 0.00001721 | 7.20 | 10.70 | | |
| Antibody 07 | 0.0169 | 6.26 | 8.62 | | |

### Example 9 Evaluation of In Vivo Anti-Tumor Efficacy of Chimeric Antibodies

The in vivo anti-tumor efficacy of antibody 01 and antibody 04 was evaluated in the humanized mouse model of tumor C57BL/6-hTNFR2 transplanted from mouse colon cancer cell line MC38.

The mouse colon cancer cell MC38 were cultured in single layer in vitro in RPMI1640 medium supplemented with 10% fetal bovine serum and 2 mM glutamine at 37° C. and 5% CO₂. It was routinely digested and passaged by trypsin-EDTA twice a week. When the cell saturation was 80-90%, cells were collected, counted and inoculated. 0.1 mL (5×10⁵ cells) of MC38 cells were subcutaneously inoculated into the right back of each mouse. When the average tumor volume reached 64 mm³, administration was started in groups.

Antibody 01 and antibody 04 were administered intraperitoneally to the tumor-bearing mice twice weekly at 2.0 mg/kg each time, and the same dose of human IgG was administered as a control for a total of 5 times.

The health and death of the animals were monitored every day. Routine examinations include observation of the effects of tumor growth and drug treatment on the animals' daily behavior performance such as behavioral activities, food and water intake (visual observation only), weight changes (weight measurements twice times a week), physical signs or other abnormal conditions. The number of deaths and side effect of animals in each group were recorded.

The experimental index was to investigate whether the tumor growth was inhibited, delayed or cured. The tumor diameter was measured with a vernier caliper three times a week. The calculation formula of tumor volume was: V=0.5axb², wherein a and b represented the long and short diameter of the tumor, respectively. The tumor suppressive efficacy was evaluated by TGI (%) or the relative tumor proliferation rate T/C (%). TGI (%) reflected the tumor growth inhibition rate. Calculation of TGI (%) was: TGI (%)=[(1−(Average tumor volume at the end of a certain treatment group−Average tumor volume at the beginning of the treatment group))/(Average tumor volume at the end of treatment in the solvent control group−Average tumor volume at the start of the treatment in the solvent control group)]×100%. Calculation of the relative tumor proliferation rate T/C (%) was: T/C %=T_{RTV}/C_{RTV}×100% (T_{RTV}: treatment group RTV; C_{RTV}: negative control group RTV). Relative tumor volume (RTV) was calculated based on the results of the tumor measurements. The calculation formula was RTV=Vt/V0, where V0 was the average tumor volume measured during group administration (that was, d0), and Vt was the average tumor volume during a certain measurement. T_{RTV} and C_{RTV} were taken from the same day.

The tumor growth curves of mouse colon cancer cell line MC38 transplantation tumor model loaded mice given human IgG control, antibody 01, antibody 04, respectively, are shown in FIG. 5, where the horizontal coordinates indicate the number of days since the start of the treatment, and the vertical coordinates indicate the tumor volume. As shown in FIG. 5, antibody 01 and antibody 04 show better in vivo anti-tumor efficacy than BMK6.

### Example 10 Humanization of Antibodies

The VHH sequences were compared with libraries of known human ethnographic lineage sequences on the NCBI website (https://www.ncbi.nlm.nih.gov/igblast/). The databases used were the IMGT human VH gene. For antibody VHH, human germline IGVH3-23 was selected as the acceptor sequence, and the human light chain IGKJ4 (allele 1) joining region (J gene) was selected from the human joining region sequences compiled at IMGT®, the international ImMunoGeneTics Information System® http://www.imgt.org. The CDRs were determined according to the AbM definition. So that the human germline frame positions were changed to the corresponding parental mouse sequences to optimize the binding of the humanized antibodies. By the above method, 5 humanized antibodies were obtained.

TABLE 6
Humanized Antibodies and Sequence Numbers

| Antibody | Sequence number |
|---|---|
| Antibody 17 | SEQ ID NO: 68 |
| Antibody 18 | SEQ ID NO: 72 |
| Antibody 19 | SEQ ID NO: 76 |
| Antibody 20 | SEQ ID NO: 80 |
| Antibody 21 | SEQ ID NO: 84 |

### Example 11 Assay of Binding Activity of the Humanized Antibodies

(1) Coating: Human-TNFR2-His was diluted to 0.5 μg/mL with coating solution (1×PBS, pH 7.4), and coated into a 96-well enzyme labeled plate at 100 μL/well at 4° C. overnight. The coating solution was poured off, and the plate was washed with 1×PBST at 300 μL per well, washed with a plate cleaning instrument for 4 times, and patted dry on sheet paper.

(2) Blocking: it was blocked with 3% skimmed milk powder at 300 μL/well, and incubated at 37° C. for 1 h. The blocking solution was poured off, washed by a plate cleaning instrument for 4 times, and patted dry on sheet paper.

(3) Sample dilution: the reference product and the test product were diluted with 3% skimmed milk powder to 10 μg/mL, and this was used as the initial concentration for 3-fold dilution with a total of 11 gradients of dilution, and another one blank well was set up, and only diluent was added. It was incubated at 100 μL/well at 37° C. for 1 h. Liquid in the wells was discarded, washed by a plate cleaning instrument for 4 times, and patted dry on the sheet paper.

(4) Addition of enzyme-labeled secondary antibody: Peroxidase-conjugated AffiniPure F(ab′)2 Fragment Goat Anti-Human IgG was diluted with 3% skimmed milk powder at 1:20,000. It was incubated at 100 μL/well at 37° C. for 1 h. It was washed by a plate cleaning instrument for 6 times and patted dry on sheet paper.

(5) Color development: TMB color development solution was added at 100 μL/well and wrapped with aluminium foil, and the color was developed at 37° C. without light for 8 min.

(6) Termination of color development: terminal solution 1M HCl was added to terminate the color development reaction at 100 μL/well.

(7) Reading at 450 nm on the enzyme labeler.

As shown in Table 7, the humanized antibodies have equivalent or better binding activity compared with the corresponding chimeric antibodies, indicating that the antibodies can maintain high binding activity after humanization.

TABLE 7
Assay of binding activity
of the humanized antibodies

| Antibody | EC50 (μg/mL) |
|---|---|
| Antibody 1 | 0.02555 |
| Antibody 17 | 0.000823 |
| Antibody 18 | 0.01157 |
| Antibody 19 | 0.00306 |
| Antibody 20 | 0.00613 |
| Antibody 21 | 0.01735 |

### Example 12 Affinity Assay of the Humanized Antibodies

Equipment: OCTET Red96e (Fortebio).
Sensor: AHC.

### (1) Experimental Setup:

Sensor preparation: the AHC sensor was soaked in 0.02% PBST (0.02% Tween 20, pH 7.4, 1×PBS) as buffer for 600 s prior to use to remove the sucrose covering the sensor surface.

### (2) Immobilization and Capture:

After equilibration of the AHC sensor with 0.02% PBST (0.02% Tween 20, pH 7.4, 1×PBS) as buffer for 60 s, the TNFR2 antibody in the sample plate was immobilized for 300 s, and the second equilibration was performed for 180 s. 100 nM of human TNFR2-His protein was bound with the TNFR2 antibody for 300 s and then dissociated for 600 s. After dissociation, 10 mM glycine (pH 2.0) was used as regeneration buffer to regenerate for 30 s.

### (3) Regeneration:

The sensor was regenerated with 10 mM glycine (pH 2.0).

### (4) Data Analysis:

As shown in Table 8, the humanized antibodies have equivalent or better binding activity compared with the corresponding antibodies, indicating that the humanized antibodies can maintain a high affinity.

TABLE 8
Affinity assay of the humanized antibodies

| | Affinity KD | Affinity | Affinity |
|---|---|---|---|
| | (single | Kon | Kdis |
| | concen- | (multiple | (multiple |
| Antibody | tration) | concentrations) | concentrations) |
| Antibody 1 | 7.94E−10 | 2.94E+05 | 2.33E−04 |
| Antibody 17 | 1.10E−09 | 2.68E+05 | 2.95E−04 |
| Antibody 18 | 1.09E−09 | 2.76E+05 | 2.99E−04 |
| Antibody 19 | 1.03E−09 | 2.67E+05 | 2.76E−04 |
| Antibody 20 | 6.49E−09 | 4.86E+05 | 3.15E−03 |
| Antibody 21 | 5.50E−09 | 5.02E+05 | 2.76E−03 |

1. An anti-TNFR2 antibody or antigen-binding fragment thereof, wherein the antibody or antigen-binding fragment thereof comprises complementary determining regions CDR1, CDR2 and CDR3, wherein,
(01) CDR1 is an amino acid sequence selected from any one of SEQ ID NOs: 2, 6, 10, 14, 18, 22, 26, 30, 34, 38, 42, 46, 50, 54, 58, 62, 69, 73, 77, 81, 85; or a sequence having at least 80%, 85%, 90%, 91%, 92%, 93%, 94%, 95%, 96%, 97%, 98%, 99% or more identity to an amino acid sequence of any one of SEQ ID NOs: 2, 6, 10, 14, 18, 22, 26, 30, 34, 38, 42, 46, 50, 54, 58, 62, 69, 73, 77, 81, 85; or an amino acid sequence having one or more conserved amino acid mutations compared with any amino acid sequence of SEQ ID NOs: 2, 6, 10, 14, 18, 22, 26, 30, 34, 38, 42, 46, 50, 54, 58, 62, 69, 73, 77, 81, 85;
(02) CDR2 is an amino acid sequence selected from any one of SEQ ID NOs: 3, 7, 11, 15, 19, 23, 27, 31, 35, 39, 43, 47, 51, 55, 59, 63, 70, 74, 78, 82, 86; or a sequence having at least 80%, 85%, 90%, 91%, 92%, 93%, 94%, 95%, 96%, 97%, 98%, 99% or more identity to an amino acid sequence of any one of SEQ ID NOs: 3, 7, 11, 15, 19, 23, 27, 31, 35, 39, 43, 47, 51, 55, 59, 63, 70, 74, 78, 82, 86; or an amino acid sequence having one or more conserved amino acid mutations compared with any amino acid sequence of SEQ ID NOs: 3, 7, 11, 15, 19, 23, 27, 31, 35, 39, 43, 47, 51, 55, 59, 63, 70, 74, 78, 82, 86; and
(03) CDR3 is an amino acid sequence selected from any one of SEQ ID NOs: 4, 8, 12, 16, 20, 24, 28, 32, 36, 40, 44, 48, 52, 56, 60, 64, 71, 75, 79, 83, 87; or a sequence having at least 80%, 85%, 90%, 91%, 92%, 93%, 94%, 95%, 96%, 97%, 98%, 99% or more identity to an amino acid sequence of any one of SEQ ID NOs: 4, 8, 12, 16, 20, 24, 28, 32, 36, 40, 44, 48, 52, 56, 60, 64, 71, 75, 79, 83, 87; or an amino acid sequence having one or more conserved amino acid mutations compared with any amino acid sequence of SEQ ID NOs: 4, 8, 12, 16, 20, 24, 28, 32, 36, 40, 44, 48, 52, 56, 60, 64, 71, 75, 79, 83, 87.

2. The antibody or antigen-binding fragment thereof of claim 1, wherein,
(01) the complementary determining regions CDR1, CDR2 and CDR3 consist of the amino acid sequences SEQ ID NO: 2, SEQ ID NO: 3 and SEQ ID NO: 4, respectively;
(02) the complementary determining regions CDR1, CDR2 and CDR3 consist of the amino acid sequences SEQ ID NO: 6, SEQ ID NO: 7 and SEQ ID NO: 8, respectively;
(03) the complementary determining regions CDR1, CDR2 and CDR3 consist of the amino acid sequences SEQ ID NO: 10, SEQ ID NO: 11 and SEQ ID NO: 12, respectively;
(04) the complementary determining regions CDR1, CDR2 and CDR3 consist of the amino acid sequences SEQ ID NO: 14, SEQ ID NO: 15 and SEQ ID NO: 16, respectively;
(05) the complementary determining regions CDR1, CDR2 and CDR3 consist of the amino acid sequences SEQ ID NO: 18, SEQ ID NO: 19 and SEQ ID NO: 20, respectively;
(06) the complementary determining regions CDR1, CDR2 and CDR3 consist of the amino acid sequences SEQ ID NO: 22, SEQ ID NO: 23 and SEQ ID NO: 24, respectively;
(07) the complementary determining regions CDR1, CDR2 and CDR3 consist of the amino acid sequences SEQ ID NO: 26, SEQ ID NO: 27 and SEQ ID NO: 28, respectively;
(08) the complementary determining regions CDR1, CDR2 and CDR3 consist of the amino acid sequences SEQ ID NO: 30, SEQ ID NO: 31 and SEQ ID NO: 32, respectively;
(09) the complementary determining regions CDR1, CDR2 and CDR3 consist of the amino acid sequences SEQ ID NO: 34, SEQ ID NO: 35 and SEQ ID NO: 36, respectively;
(10) the complementary determining regions CDR1, CDR2 and CDR3 consist of the amino acid sequences SEQ ID NO: 38, SEQ ID NO: 39 and SEQ ID NO: 40, respectively;
(11) the complementary determining regions CDR1, CDR2 and CDR3 consist of the amino acid sequences SEQ ID NO: 42, SEQ ID NO: 43 and SEQ ID NO: 44, respectively;
(12) the complementary determining regions CDR1, CDR2 and CDR3 consist of the amino acid sequences SEQ ID NO: 46, SEQ ID NO: 47 and SEQ ID NO: 48, respectively;
(13) the complementary determining regions CDR1, CDR2 and CDR3 consist of the amino acid sequences SEQ ID NO: 50, SEQ ID NO: 51 and SEQ ID NO: 52, respectively;
(14) the complementary determining regions CDR1, CDR2 and CDR3 consist of the amino acid sequences SEQ ID NO: 54, SEQ ID NO: 55 and SEQ ID NO: 56, respectively;
(15) the complementary determining regions CDR1, CDR2 and CDR3 consist of the amino acid sequences SEQ ID NO: 58, SEQ ID NO: 59 and SEQ ID NO: 60, respectively;
(16) the complementary determining regions CDR1, CDR2 and CDR3 consist of the amino acid sequences SEQ ID NO: 62, SEQ ID NO: 63 and SEQ ID NO: 64, respectively;
(17) the complementary determining regions CDR1, CDR2 and CDR3 consist of the amino acid sequences SEQ ID NO: 69, SEQ ID NO: 70 and SEQ ID NO: 71, respectively;
(18) the complementary determining regions CDR1, CDR2 and CDR3 consist of the amino acid sequences SEQ ID NO: 73, SEQ ID NO: 74 and SEQ ID NO: 75, respectively;
(19) the complementary determining regions CDR1, CDR2 and CDR3 consist of the amino acid sequences SEQ ID NO: 77, SEQ ID NO: 78 and SEQ ID NO: 79, respectively;
(20) the complementary determining regions CDR1, CDR2 and CDR3 consist of the amino acid sequences SEQ ID NO: 81, SEQ ID NO: 82 and SEQ ID NO: 83, respectively; or
(21) the complementary determining regions CDR1, CDR2 and CDR3 consist of the amino acid sequences SEQ ID NO: 85, SEQ ID NO: 86 and SEQ ID NO: 87, respectively.

3. The antibody or antigen-binding fragment thereof of claim 2, wherein,
the sequence of the antibody or antigen-binding fragment thereof is selected from the amino acid sequence of any one of SEQ ID NO: 1, 5, 9, 13, 17, 21, 25, 29, 33, 37, 41, 45, 49, 53, 57, 61, 68, 72, 76, 80, 84,
or a sequence having at least 80%, 85%, 90%, 91%, 92%, 93%, 94%, 95%, 96%, 97%, 98%, 99% or more identity to the amino acid sequence given in SEQ ID NO: 1, 5, 9, 13, 17, 21, 25, 29, 33, 37, 41, 45, 49, 53, 57, 61, 68, 72, 76, 80, 84,
or an amino acid sequence having one or more conserved amino acid mutations as compared with any of the amino acid sequences of SEQ ID NO: 1, 5, 9, 13, 17, 21, 25, 29, 33, 37, 41, 45, 49, 53, 57, 61, 68, 72, 76, 80, 84.

4. The antibody or antigen-binding fragment thereof of claim 3, wherein the antibody or antigen-binding fragment thereof is a single-domain antibody VHH chain.

5. (canceled)

6. (canceled)

7. The antibody or antigen-binding fragment thereof of claim 4, wherein the single-domain antibody VHH chain further comprises an immunoglobulin Fc region, and the immunoglobulin Fc region is selected from the group consisting of: IgG1, IgG2, IgG3, and IgG4.

8. The antibody or antigen-binding fragment thereof of claim 4, wherein the single-domain antibody VHH chain further comprises the amino acid sequence of the immunoglobulin constant region SEQ ID NO: 65.

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

22. (canceled)

22. (canceled)

23. (canceled)

24. (canceled)

25. The antibody or antigen-binding fragment thereof of claim 2, wherein the antibody or antigen-binding fragment thereof comprises camelid, chimeric, humanized or fully human.

26. (canceled)

27. (canceled)

28. (canceled)

29. A pharmaceutical composition comprising the antibody or antigen-binding fragment thereof claim 1 and a pharmaceutically acceptable carrier, diluent or excipient.

30. The pharmaceutical composition of claim 29, wherein the composition further comprises an additional therapeutic agent.

31. The pharmaceutical composition of claim 30, wherein the additional therapeutic agent is an immunotherapeutic agent.

32. (canceled)

33. Use of the antibody or antigen-binding fragment thereof of claim 1 in the following (a), (b), (c), (d), (e) or (f):
(a) preparation of a drug that induces apoptosis in Treg cells;
(b) preparation of a drug that has Treg proliferation inhibitory properties;
(c) preparation of a drug that blocks the TNF-TNFR2 tumor signaling pathway;
(d) preparation of a drug that promotes the proliferation and differentiation and/or biological activity of Teff cells;
(e) preparation of a drug for the treatment of autoimmune diseases; or
(f) preparation of a drug that targets tumor cells for killing.

34. The use of claim 33, wherein the tumor is selected from: ovarian cancer, melanoma, prostate cancer, intestinal cancer, gastric cancer, esophageal cancer, breast cancer, lung cancer, renal cancer, pancreatic cancer, uterine cancer, hepatocellular carcinoma, bladder cancer, cervical cancer, oral cancer, brain cancer, testicular cancer, skin cancer, colorectal cancer, malignant glioma, thyroid cancer, or a related tumor.
